(19) United States
(12) Patent Application Publication
(10) Pub. No.: US 2025/0018336 A1
(43) Pub. Date: Jan. 16, 2025

(54) BIO-DEGRADABLE POROUS BONDED-GRAIN MONOLITHIC CO2 SCRUBBER FOR RESPIRATION DEVICES

(71) Applicant: Tesseron Ltd., Key Largo, FL (US)

(72) Inventors: Forrest P. Gauthier, Homestead, FL (US); Jeffrey E. Bozanic, Fountain Valley, CA (US)

(73) Assignee: Tesseron Ltd., Key Largo, FL (US)

(21) Appl. No.: 18/772,904

(22) Filed: Jul. 15, 2024

Related U.S. Application Data
(60) Provisional application No. 63/526,997, filed on Jul. 15, 2023.

(51) Int. Cl. B01D 53/82; A61M 16/22; A62B 23/02; B01D 53/62; B01J 20/04; B01J 20/28; B01J 20/30; B01J 20/32

(52) U.S. Cl. CPC B01D 53/82; A61M 16/22; A62B 23/02; B01D 53/62; B01J 20/041; B01J 20/28042; B01J 20/3007; B01J 20/3042; B01J 20/3204; B01J 20/3268; B01D 2251/302; B01D 2251/304; B01D 2251/306; B01D 2251/404; B01D 2251/604; B01D 2253/342; B01D 2257/504

(57) ABSTRACT

The present disclosure relates to a biodegradable porous bonded-grain monolithic CO2 scrubber for respiration devices. The scrubber comprises a monolithic body of bonded grains, including calcium hydroxide, sodium hydroxide, lithium hydroxide, and potassium hydroxide, to act as CO2 absorbents. The body features enclosed gas passages facilitating gas flow and CO2 absorption. The grains are bonded via adhesion, powder polymer, or heat sintering, and the structure includes hydrophobic additives to reduce caustic solution formation. The scrubber can be manufactured through extrusion or additive manufacturing, with various passage shapes and sizes. The monolithic body may include biodegradable polymers for environmentally sound disposal.

### CROSS REFERENCE

This application claims the benefit of U.S. Provisional Application No. 63/526,997, filed Jul. 15, 2023, the disclosure of which is incorporated herein by reference.

### FIELD

The disclosure generally relates to CO2 scrubbers. More particularly, the disclosure relates to CO2 scrubber blocks, CO2 scrubber systems, methods of permanently sequestering CO2 within a CO2 scrubber, and methods for manufacturing a CO2 scrubber block.

### BACKGROUND

Human respiration has gaseous impurities or contaminants which need removal in closed circuit and semi-closed circuit respiration breathing systems. For example, closed circuit and semi-closed circuit respiration devices such as scuba, rescue and medical rebreathers have a need to remove excessive CO2 in the breathing loop to safe and healthy levels. The removal of unwanted gaseous components like CO2 can be accomplished using a variety of methods such as cryogenic separation, swing absorption, or the use of chemical or mineral CO2 absorbents. These technologies are well understood and widely used in respiration, medical and industrial applications.

CO2 scrubbers using chemicals most commonly use calcium hydroxide (Ca(OH)2), sodium hydroxide (NaOH), lithium hydroxide (LiOH) and or potassium hydroxide (KOH) which are chemical compounds that are synthetically produced or obtained through chemical reactions. They are composed of specific elements (calcium, sodium, lithium, oxygen, and hydrogen) and have their own distinct chemical properties.

Some chemical CO2 scrubbers use loose adsorbent grains, extruded absorbent pellets and extruded absorbent sheets which are enclosed in a container, wound in a coil or hung as sheets of material composed of any variety of chemicals commonly used for CO2 absorption such as Ca(OH)2, LiOH, NaOH, KOH either as sole constituents or in combination.

However, previous CO2 scrubbers may have irregular absorbent packing, granular absorbent shifting, gas flow channeling, telescoping or displacement of extruded sheets, mechanically induced absorbent dust production and the potential of a “caustic cocktail” of free caustics solutions from water intrusion, condensation or as byproduct of the absorption chemical reaction.

In some scrubbers the polymers used to house, frame or bond the grains together are PTFE, ABS or other non-biodegrading polymers, which remain as polymer chains in nature. This creates a recycle or environmental concern for the disposal of the scrubbers after use. Some CO2 scrubbers often suffer from issues such as inefficient CO2 capture, instability of the captured CO2, and environmental concerns due to non-biodegradable materials. For instance, traditional scrubbers using calcium hydroxide (Ca(OH)2) often require frequent replacement due to the re-release of CO2 during regeneration. Moreover, many scrubbers rely on plastic housings that contribute to environmental pollution. There is a clear need for a more efficient, stable, and environmentally friendly CO2 scrubber that can provide permanent sequestration of carbon while minimizing ecological impact.

### BRIEF SUMMARY OF SELECTED EXAMPLES

A porous monolithic body of bonded grains of a calcium hydroxide (Ca(OH)2), sodium hydroxide (NaOH), lithium hydroxide (LiOH), and/or potassium hydroxide (KOH) with the addition of hydrophobic and non-hydrophobic additives as a CO2 absorbent structure. The body is formed into a three dimensional shape with one or more enclosed gas passages. The preferred embodiment for use in a rebreather apparatus has a multitude of gas passages engineered to achieve the required absorbent capacity and gas flow. In the present disclosure blocks of 58.5×60×50.5 mm are formed with 325 hexagonal shaped gas passages, each hexagonal passage having a apothem of 1.057 mm. The absorbent grains are bonded together using an adhesive, powder polymer or by heat sintering. The block is further coated or embedded with a gas permeable hydrophobic material. This material may be but is not limited to PTFE grains, resin, liquid, low soluble lipids and other hydrophobic nano-particles.

The absorbent body may be produced by extrusion through a pressure extrusion process and die, or by additive manufacturing (3-D printing). The additive manufacturing process may utilize a powder bed-based system or laser sintering, with or without added polymers, and may use a thermoset system or polymer. The absorbent body will feature one or more enclosed gas passages, which may be uniform polygons in cross-section or irregularly truncated at the cartridge margins. These passages may allow for linear gas flow from one side to the other, or be curvilinear, allowing gas flow to enter from one direction and exit in another. The passage shapes may be circular, triangular, rectilinear, pentagonal, hexagonal, star-shaped, or other forms, with multiple shapes used to maximize adsorbent efficacy, structural strength, raw material efficiency, and other factors for specific applications. The shapes and sizes of the passages will vary to achieve desired flow speeds, residence times, pressure drops, boundary layer flows, moisture content, and gas contaminant removal efficacy.

The absorbent body may include structural extrusions to increase strength and rigidity, minimizing damage during manufacture, shipping, or use. The assembly may also include a co-molded or over-molded exterior frame. Polymers or other substances may be incorporated to stabilize shape, increase durability, enhance hydrophobic properties, stabilize adsorbent grains or powders, or for other purposes. The absorbent body assembly may include a co-molded handling device.

The grain stabilizer in the absorbent body may be made of ePTFE polymer, LPA polymer, PBSA (polybutylene succinate-co-adipate) polymer (for use with thermoset additive processes), alginate, or cellulose, with thermoform options like PHA (polyhydroxyalkanoates) or PLA (polylactic acid). The polymer used in the absorbent body may be biodegradable, such as LPA, alginate, or cellulose, allowing for environmentally sound disposal, including ocean disposal where it may promote de-acidification.

The production methodology may include block extrusion with binding polymers and oils, oil separation and removal with vacuum and solvents, drying of the block after fabrication to control hydration, hydration of the block to activate chemical reactions, powder removal by blowing, and chemical vapor deposition (CVD) of hydrophobic coatings, such as PTFE, TFE, PFA, or PECVD.

One embodiment is a biodegradable porous bonded-grain monolithic CO2 scrubber for respiration devices. It comprises a porous monolithic body made of bonded grains of at least one of calcium hydroxide, sodium hydroxide, lithium hydroxide, and potassium hydroxide, to act as the CO2 absorbent chemicals. Additionally, the scrubber includes a hydrophobic additive configured to reduce the formation of free caustic solutions. The monolithic body includes a plurality of enclosed gas passages, each configured to facilitate gas flow and CO2 absorption. The bonded grains within the monolithic body are bonded together using at least one of adhesion, powder polymer, and heat sintering.

Another embodiment is a method for manufacturing a biodegradable porous bonded-grain monolithic CO2 scrubber. The method includes extruding a monolithic body of bonded CO2 absorbent grains. Additionally, the method includes embedding the body with hydrophobic materials configured to reduce the formation of caustic solution. Even further, the method includes defining gas passages within the monolithic body for CO2 absorption efficiency.

Various aspects of this disclosure will become apparent to those skilled in the art from the following detailed description of the preferred embodiments, when read in light of the accompanying drawings.

### BRIEF DESCRIPTION OF FIGURES

FIG. 1 is first view of a first porous bonded-grain monolithic CO2 scrubber block including pre-engineered gas passages.

FIG. 2 is first view of a second porous bonded-grain monolithic CO2 scrubber block including pre-engineered gas passages.

FIG. 3 is second view of a first porous bonded-grain monolithic CO2 scrubber block including pre-engineered gas passages.

FIG. 4 is second view of a first porous bonded-grain monolithic CO2 scrubber block including pre-engineered gas passages.

FIG. 5 is a first portion of the septum designed to separate the passages of the second porous bonded-grain monolithic CO2 scrubber block.

FIG. 6 is a second portion of the septum designed to separate the passages of the second porous bonded-grain monolithic CO2 scrubber block.

FIG. 7 is a first view of a monolithic CO2 scrubber block disposed in a frame.

FIG. 8 is a second view of a monolithic CO2 scrubber block disposed in a frame.

FIG. 9 is a first view of an extrusion die.

FIG. 10 is a second view of an extrusion die.

FIG. 11 is a closeup view of a portion of the extrusion die of FIG. 10.

FIG. 12 is a comparative table illustrating the required, micropore, and current implementation values for various parameters including adsorbent mass, gas channel size, channel perimeter, number of channels per block, channel surface area, gas surface area, and gas flow rates.

### DETAILED DESCRIPTION OF SELECTED EXAMPLES

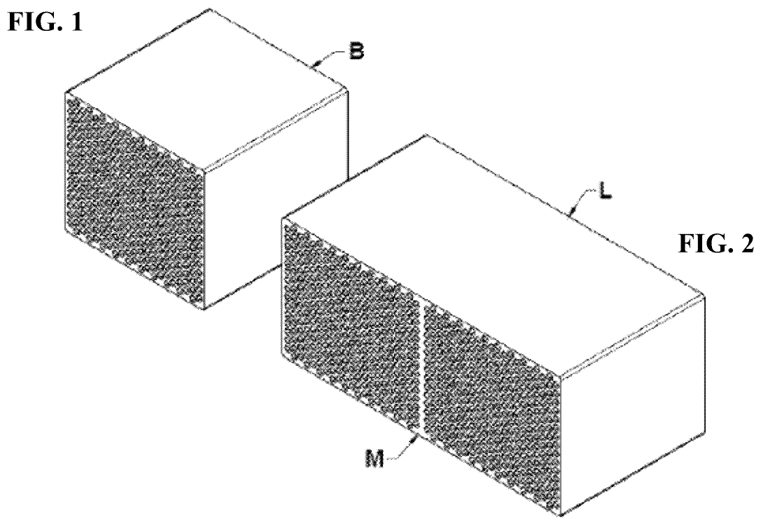

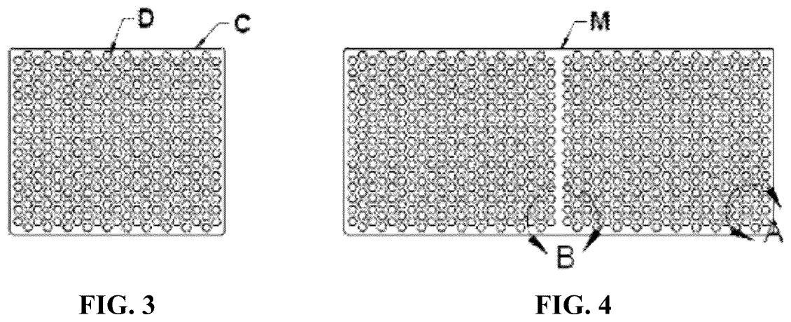

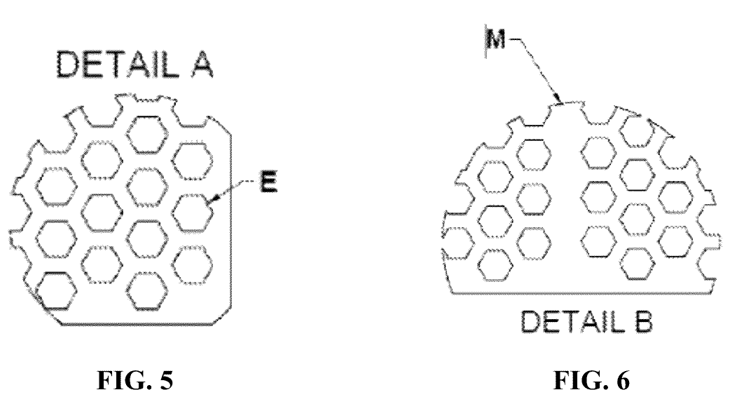

FIGS. 1, 2, 3, and 4 present various examples of a porous bonded-grain monolithic CO2 scrubber block. FIGS. 5 and 6 present a septum configured to separate the passages of a porous bonded-grain monolithic CO2 scrubber block.

One embodiment is a novel design, method and application of chemicals used for CO2 absorption, primarily calcium hydroxide (Ca(OH)2), sodium hydroxide (NaOH), lithium hydroxide (LiOH) and or potassium hydroxide (KOH) with the addition of trace polymer additives and disclosed processes to shape the chemical grains into a porous bonded-grain monolithic block with pre-engineered CO2 absorbent specifications for the block. The block further has one or more pre-engineered bore or through-bore passages, which may be curved or linear, for gas flow. The term bore, through-bore or passage meaning a hole or passage that extends through a material, enclosed similar to a pipe, with open ends. It conveys the design of a well-defined, continuous, and enclosed hole fluidly coupled from one face to another other, yet does not mean the passage must be linear. The block may have passages for a single pass scrubber as shown in FIG. 1 or the block may have two sets of passages for the bidirectional flow of gas FIG. 2 with a septum designed to separate the passages and therefore the flow of the bidirectional flow of gas as shown in FIG. 2.

The present disclosure presents an improvement in respiration CO2 scrubber design by, among other things, using a porous bonded-grain monolithic block of grains of CO2 scrubber material with a pre-engineered quantity of material and one or more pre-engineered passages in the block for gas or fluid to convey through. This represents a significant improvement over existing designs when used by novice operators of the scrubber unit. The CO2 scrubber utilizes a unique combination of calcium hydroxide (Ca(OH)2), sodium hydroxide (NaOH), lithium hydroxide (LiOH), and potassium hydroxide (KOH) as the primary sorbent materials. These hydroxides may be chosen for their high reactivity with CO2 and their ability to form stable carbonate compounds. The scrubber also incorporates biodegradable polymers, such as polylactic acid (PLA) and alginate, which serve as binders and structural components. These materials not only enhance the environmental sustainability of the scrubber but also improve its overall performance and durability.

During the absorption phase, CO2 reacts with calcium hydroxide (Ca(OH)2) to form calcium carbonate (CaCO3) and water (H2O). This exothermic reaction not only sequesters CO2 permanently but also releases heat that can be utilized to enhance the system's efficiency. The stable nature of CaCO3 ensures that CO2 remains fixed within the scrubber material, preventing re-release during regeneration. The heat generated from this exothermic reaction is redirected within the system to maintain optimal temperatures for further CO2 absorption. This thermal self-sufficiency enhances the scrubber's operational efficiency and reduces the need for additional external energy inputs. The scrubber also features robust sealing mechanisms and pressure controls to prevent any accidental release of CO2, ensuring reliable and long-term sequestration.

Previous CO2 scrubber designs have utilized granular particles, extruded pellets, and extruded sheets. However, these prior methods may exhibit inconsistent performance and required skilled operators to determine quantity of material to use, the proper assembly of the chemical into a container and the proper handling and use of the chemical to prevent shifting or other movement of the material. The current disclosure addresses these limitations by providing a pre-engineered porous bonded-grain monolithic block of CO2 absorbent grains having a consistent and predictable performance, even when assembled and or used by novice individuals.

The present disclosure includes a monolithic scrubber material which incorporates grain-embedded or surface coatings of gas permeable hydrophobic compounds or nano-particles which impart resistance to the formation of free caustic solutions. This feature reduces the release of unwanted caustic solutions from the scrubber block and or breathing loop. The hydrophobic additives, compounds or nano-particles are added to the scrubber material in a minimal mass to resist the formation of free caustic solutions forming and escaping from the block while allowing gas to permeate the grains of the scrubber chemical.

The present disclosure includes gas flows through passages in the block are deterministic as are the surface areas of the gas/chemical interaction. Gas which enters the fixed passages in the monolithic block flow through the passage length using a deterministic surface area while partial pressure diffusion of that surface area allows the transfer of gas into the scrubber material. In some embodiments of the current disclosure the entry and exit from the passages may be on opposite faces of the block, while in other embodiments the passages may be in non-linear orientations.

The present disclosure provides a solution to the shortcomings of prior CO2 scrubbers by employing a novel chemical reaction to permanently sequester CO2 within the sorbent material. The reaction between carbon dioxide and calcium hydroxide forms calcium carbonate, a stable compound, ensuring that the CO2 is not released back into the environment. During the absorption phase, CO2 reacts with calcium hydroxide (Ca(OH)2) to form calcium carbonate (CaCO3) and water (H2O). This exothermic reaction not only sequesters CO2 permanently but also releases heat that is utilized to enhance the system's efficiency. The stable nature of CaCO3 ensures that CO2 remains fixed within the scrubber material, preventing re-release during regeneration. The CO2 molecules are drawn into the scrubber where they encounter the calcium hydroxide (Ca(OH)2) sorbent. The chemical reaction between CO2 and Ca(OH)2 is efficient, resulting in the formation of calcium carbonate (CaCO3) and water. The heat generated from this exothermic reaction is redirected within the monolithic block to maintain and stimulate optimal temperatures for further carbon absorption reactions. This intrinsic heat source reduces the need for additional external energy inputs, thereby improving the energy efficiency of the scrubber. Calcium carbonate (CaCO3) is a stable, non-volatile compound under a wide range of environmental conditions. This stability ensures that once CO2 is converted into CaCO3, it remains permanently sequestered within the scrubber material. The formation of CaCO3 eliminates the risk of carbon re-release into the environment, making this method highly effective for long-term carbon sequestration. This characteristic is particularly beneficial for applications requiring reliable and enduring carbon capture. By utilizing the heat produced during the exothermic reaction, the system can sustain a controlled thermal environment conducive to continuous CO2 absorption. This thermal self-sufficiency enhances the scrubber's operational efficiency. The reliance on the exothermic reaction for heat reduces the system's overall energy demands, making it more sustainable and cost-effective in the long run.

In the present disclosure, the CO2 scrubber blocks may be formed using various techniques taking advantage of economies of scale, desired shape or other product requirements. Forming techniques such as soft material extrusion dies, slurry or compression casting, or additive processes may all be successfully used to meet the end product requirements. The processes of the disclosure result in a deterministic quantity of scrubber material, a deterministic gas flow through the engineered gas passages and a robust block of chemical with predictable performance.

This present disclosure differs from previous extruded scrubber products which produce a pellet or a sheet which do not contain a predetermined number or size of passages for gas or fluid or a pre-engineered performance result.

The disclosure further differs from ceramic or other catalytic converters as the monolithic block is made of porous bonded grains of calcium hydroxide (Ca(OH)2), sodium hydroxide (NaOH), lithium hydroxide (LiOH), and/or potassium hydroxide (KOH) with the addition of hydrophobic additives.

To enhance the structural integrity and durability of the monolithic scrubber material in manufacturing methods such as extrusion and some forms of additive manufacturing small amounts of polymers may be incorporated as binders to the porous grain structure as described in McKenna U.S. Pat. No. 5,964,221 and Gauthier 2020/0001956. These binders are one method to bind the grains to allow the formation of the monolithic block, improve the material's resistance to degradation and facilitate ease of handling during manufacturing and use. The polymer used may be PTFE, which functionally provides hydrophobic resistance for the adsorbent material. In other embodiments, the polymer may be PLA or other biodegradable polymers, either as the sole material or in combination with a gas-permeable water resistant layer which may optionally be applied to the block after fabrication. Seaweed and algae-based PLA may be derived from marine biomass, offering a sustainable and renewable alternative to traditional PLA materials. The production process involves the fermentation of seaweed or algae biomass to produce lactic acid, which is then polymerized into PLA. This method reduces reliance on land and freshwater resources and significantly lowers the carbon footprint. Seaweed and algae-based PLA retain the biodegradability and mechanical strength of conventional PLA, with additional benefits of improved moisture resistance and UV stability. In one embodiment the scrubber utilizes seaweed and algae-based PLA for various structural components, including the housing and frames. This integration ensures the entire unit is biodegradable and environmentally friendly. The PLA also serves as a coating or binding material for the sorbent grains, enhancing structural integrity and overall performance.

With laser sintering as the manufacturing method grain bonding additives are not always required and not necessary for the formation of the monolithic block, to improve the material's resistance to degradation or to facilitate ease of handling during manufacturing and use. In one embodiment of the disclosure, a CO2 laser is used to partially fuse the grains into a sinter. In this process a focused beam of laser energy is passed over a thin layer of chemical powder layer by layer fusing the particles into a porous block based on the pattern of the laser as it scans the powder layer without the need for polymer bonding agents. In another embodiment trace amounts of polylactic acid based polyester powders are incorporated in the matrix with the laser at least partially melting the polylactic acid powder to sinter the chemical grains together. While the current embodiment uses a CO2 laser a fiber laser or other lasers with appropriate characteristics could be used.

In another embodiment of additive manufacturing, a jet of the selected adhesive is sprayed onto the chemical layer by layer, again fusing the grains into a porous block in the pattern determined by the jet of adhesive.

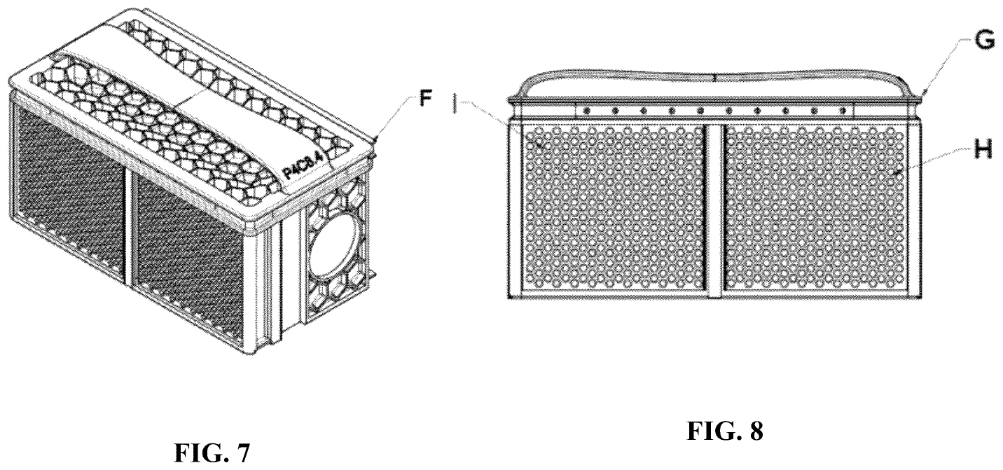

FIGS. 7 and 8 illustrate a monolithic CO2 scrubber block disposed in a frame. One or more monolithic scrubber blocks as shown in FIG. 8 may be incorporated into a holder or frames to combine one or more blocks into a pre-engineered module. See FIGS. 7 and 8. This differs from the previous CO2 scrubbers at least because the modules are pre-engineered, the individual blocks are not accessible as discrete units, the user has no control of the assembly of the holder used in the module and the entire unit is a fixed, non-adjustable assembly.

These holders facilitate the proper block alignment, block quantity alignment of the scrubber blocks. An exterior frame may be employed to increase sealing efficacy, reduce cartridge damage, or improve ease of installation. Additionally sealing mechanisms may be incorporated to reduce gas bypass or water intrusion. The frame may be a separate part or co-molded onto one or more blocks. In the current embodiment the frame is a separate part made of bio-degradable polymer and it holds two blocks.

The scrubber disclosed in 2020/0001956 includes a stack of scrubber sheets arranged in 25 layers. A septum divides the cartridge in half. The rectangular holes (channels) made by the stacked sheets are 3 mm wide and 0.65 mm tall with a perimeter 7.3 mm and a 60 mm length of the sheet. Each horizontal hole is separated by a 1 mm wall and each vertical hole is separated by a 1.35 mm wall. 25 holes are in the vertical and 14 holes are in the horizontal, giving 350 holes on each side, or 700 total for the two sets of sheets. Each hole is 3×0.65×60 mm giving a surface area of each hole of 7.3×60 mm=439.2 mm2 and a total surface area of 307,440 mm2 vs the 144,000 mm2 needed by calculation.

In the current implementation of the disclosure, about 200 g of absorbent grains are used for each 30 liters of O2 where prior designs use 654.4 g of absorbent for each 30 liters of O2.

An optimal passage size, passage shape, number of passages and thickness of the chemical web between the passages was determined. The ISO recommendation for respiration devices, ISO 23328-1:2021, recommends that at a high respiration rate of 160 LPM the inhalation pressures should not exceed 1.5 kPa (0.22 PSI), while the recommend exhale pressure not exceed 0.7 kPa (0.1 PSI).

In the current implementation, 1.22 mm hexagon passages was selected, yet this is only desirable for a specific design and engineering goal of a specific implementation.

To confirm adequate gas flow through the monolithic block, a single hexagonal gas flow channel of different sizes was tested, with sides measuring 1.0, 1.22, and 1.5 mm. Also lengths of 60 mm and 120 mm, representing gas flow distances through both a single block, and multiple blocks in series as used in the current implementation was tested. Testing was done using dry air at 1 ATM.

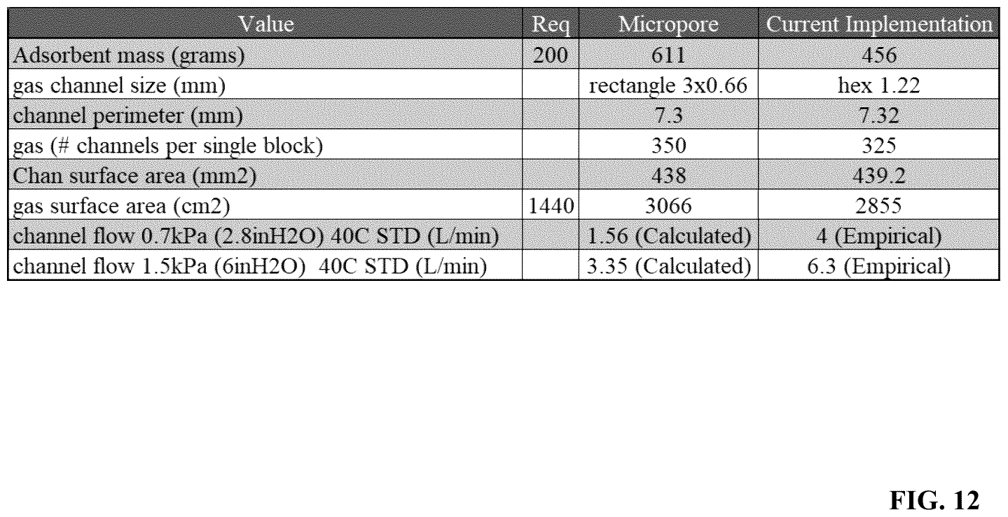

FIG. 12 presents a table that represents both calculated and empirical values developed from the testing, and as used in the current implementations of the disclosure (incorporating two monolithic blocks).

In the current implementation the monolithic block of scrubber is 58.5×60×50.5 mm giving a total volume of 177,255 mm3. Gas channel volume needs to be removed from this total volume to derive the absorbent volume. Utilizing a hexagonal channel geometry with sides of 1.22 mm as noted above, the volume of a single channel is 232 mm3, and that the total volume of the 325 gas channels in the single block is 75,406 mm3. This leaves a material volume of 101,849 mm3, with a chemical weight of approximately 228 g. The current implementation incorporates two blocks of this size, with a gas mixing chamber incorporated between them. This provides a total chemical weight of about 456 g, exceeding the calculated mass of 200 g of absorbent chemical necessary for intended use.

Adequate surface area in which gas containing CO2 or other contaminants is also necessary. Using hexagonal channels with hexagon sides of 1.22 mm and 60 mm length yields 439.2 mm2 surface area per channel. The total gas contact surface area of the 325 channels in a single block is 142,740 mm2, or 285,4809 mm2 in the current implementation utilizing two monolithic blocks in series. This exceeds the 144,000 mm2 as noted above.

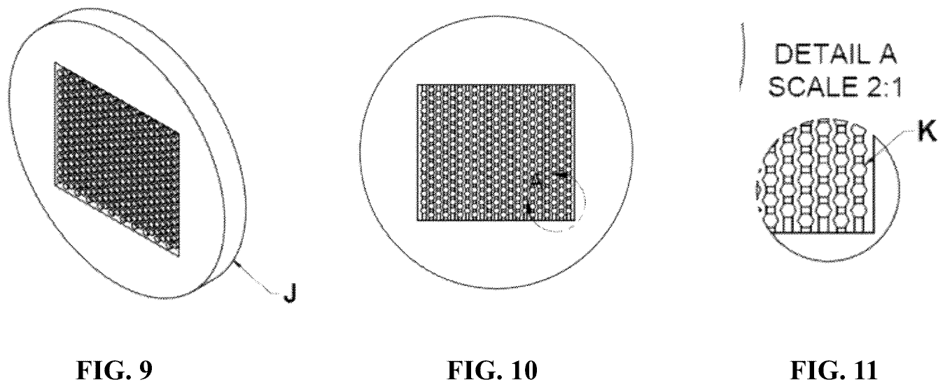

FIGS. 9, 10, and 11 are directed at an extrusion die. The monolithic block may be built using different methods based on the product requirements. For the highest volume lowest cost product pressure extrusion is the preferred method. Pressure extrusion requires a tooling die in the shape of the desired block and the desired passages. In one embodiment the current disclosure the extrusion die is an industry standard 4″ round die as shown in FIG. 9. The low pressure die was 3D printed for prototyping and extrudes a block that is 58.5×60×50.5 mm with hexagonal passages with sides of 1.22 mm and an apothem of 1.057 mm as shown in FIG. 11. This scrubber block is assembled into a polymer housing using two blocks and a gas separating septum to control the bi-direction of the gas as shown in FIG. 7. While this is the preferred embodiment, other configurations and sizes are expected for other scrubber units.

In another embodiment, a CO2 laser was used to selectively sinter the material into heat bonded layers by scanning the laser over the surface of the material, one layer at a time. In another embodiment, an adhesive binder jet based is passed over the material layer by layer bonding the grains with the adhesive. While not limited, the above methods enable the precise deposition and solidification of calcium materials into porous sinters, allowing for the creation of complex passages with non-linear gas flows. For example, monolithic blocks of calcium based scrubber material my be formed into curved passages to direct gas flows with less supporting structures, reducing or eliminating the dependence on polymer frames or other structures.

Methods of permanently sequestering CO2 within a scrubber are also provided. These methods include using a CO2 scrubber according to an embodiment to such that CO2 reacts with calcium hydroxide (Ca(OH)2) in the CO2 scrubber to form stable carbonate compounds, preventing the release of CO2 during the regeneration process.

One aspect of particular embodiments of the inventive CO2 scrubbers is inclusion of a hydrophobic coating of the block. Another aspect of particular embodiments of the inventive CO2 scrubbers is the inclusion of hydrophobic particle and or nano-particles imbedded in the grains of the block, which are different from the ingrained polymers. Another aspect of particular embodiments of the inventive CO2 scrubbers is the inclusion of elements for performing an integrated carbonation process, where absorbed CO2 reacts with mineral layers within the scrubber to form stable carbonates, enhancing the fixation of CO2 and the structural integrity of the scrubber material.

Particular embodiments of the inventive CO2 scrubbers comprise a CO2 scrubber block composed of a mixture of calcium hydroxide (Ca(OH)2), sodium hydroxide (NaOH), lithium hydroxide (LiOH), and potassium hydroxide (KOH), combined with one or more biodegradable polymers, such as polylactic acid (PLA) and alginate, providing high reactivity with CO2 and environmental sustainability.

Particular embodiments of the inventive CO2 scrubbers comprise a CO2 scrubber block featuring integrated gas passages configured to optimize gas flow and surface area contact, with structural enhancements such as hydrophobic coatings and polymer binders to prevent the formation and escape of caustic solutions.

One aspect of particular embodiments of the inventive CO2 scrubber systems is the use of a multi-stage absorption process, where CO2 is first absorbed by a primary sorbent and then subjected to secondary and tertiary chemical reactions to convert CO2 into stable compounds, ensuring no gaseous CO2 is released. Another aspect of particular embodiments of the inventive CO2 scrubber systems is the inclusion of biodegradable materials to minimize environmental impact by reducing waste and allowing for potential recycling or repurposing of the scrubber material.

One aspect of particular embodiments of the inventive methods is the inclusion of regenerating the CO2 scrubber material using the exothermic heat generated during the CO2 absorption phase to facilitate the desorption process, ensuring continuous operation and enhanced efficiency without external heating sources.

One aspect of particular embodiments of the inventive methods for permanently sequestering CO2 within a scrubber including allowing absorbed CO2 to react with calcium hydroxide (Ca(OH)2) to form stable carbonate compounds, preventing the release of CO2 during the regeneration process.

One aspect of particular embodiments of the inventive methods for manufacturing a CO2 scrubber block includes use of one or more techniques, such as extrusion, laser sintering, and additive manufacturing, to ensure consistent quality and performance of the scrubber material.

The principle and mode of operation of this disclosure have been explained and illustrated in its preferred embodiment. However, it must be understood that this disclosure may be practiced otherwise than as specifically explained and illustrated without departing from its spirit or scope.

What is claimed is:

1. A biodegradable porous bonded-grain monolithic CO2 scrubber for respiration devices, comprising:
a porous monolithic body of bonded grains of at least one of calcium hydroxide, sodium hydroxide, lithium hydroxide, and potassium hydroxide to act as a CO2 absorbent chemical; and
a hydrophobic additive configured to reduce the formation of free caustic solutions,
wherein the monolithic body defines a plurality of enclosed gas passages formed within the monolithic body, each passage of the plurality of gas passages being configured to facilitate gas flow and CO2 absorption;
wherein the bonded grains are bonded together via at least one of adhesion, powder polymer, and heat sintering.

2. The CO2 scrubber of claim 1, wherein a passage within the plurality of gas passages define a hexagon having an apothem of generally 1.057 mm.

3. The CO2 scrubber of claim 1, further comprising:
a gas-permeable hydrophobic coating composed of at least one of PTFE grains, resin, low soluble lipids, and hydrophobic nano-particles.

4. The CO2 scrubber of claim 3, wherein the gas-permeable hydrophobic coating is applied via chemical vapor deposition.

5. The CO2 scrubber of claim 1, wherein a grain stabilizer in the porous monolithic is composed of at least one of ePTFE polymer, LPA polymer, PBSA polymer, alginate, and cellulose.

6. The CO2 scrubber of claim 1, wherein a grain stabilizer in the porous monolithic is composed of at least one biodegradable polymer such as PHA and PLA.

7. The CO2 scrubber of claim 1, further comprising a septum configured to separate bidirectional gas passages within the monolithic body.

8. The CO2 scrubber of claim 1, wherein the monolithic block is approximately 58.5×60×50.5 mm.

9. A method for manufacturing a biodegradable porous bonded-grain monolithic CO2 scrubber, comprising:
extruding a monolithic body of bonded CO2 absorbent grains;
embedding the body with hydrophobic materials to reduce caustic solution formation;
defining gas passages within the body for CO2 absorption efficiency.

10. The CO2 scrubber of claim 1, wherein the polymer used in the porous monolithic block is composed of at least one of ePTFE polymer, LPA polymer, PBSA polymer, alginate, and cellulose.

11. The CO2 scrubber of claim 1, wherein the polymer used in the porous monolithic block is composed of at least one of PHA and PLA.

12. The CO2 scrubber of claim 1, further comprising an exterior frame co-molded onto the monolithic block, the frame being composed of a biodegradable polymer.

13. A biodegradable porous bonded-grain monolithic CO2 scrubber for respiration devices, comprising:
a porous monolithic body of bonded grains of at least one of calcium hydroxide, sodium hydroxide, lithium hydroxide, and potassium hydroxide to act as CO2 absorbent chemicals;
a hydrophobic additive configured to reduce the formation of free caustic solutions;
a plurality of enclosed gas passages formed within the monolithic body, each passage configured to facilitate gas flow and CO2 absorption;
wherein the heat generated from the exothermic reaction between CO2 and the CO2 absorbent chemicals is redirected within the monolithic block to maintain a predetermined optimal temperature for further CO2 absorption;
wherein the bonded grains are bonded together via at least one of adhesion, powder polymer, and heat sintering.

14. The CO2 scrubber of claim 13, wherein the exothermic reaction between CO2 and calcium hydroxide (Ca(OH)2) forms calcium carbonate (CaCO3) and water (H2O).

15. The CO2 scrubber of claim 13, further comprising biodegradable polymers such as polylactic acid (PLA) and alginate as binders and structural components to enhance the environmental sustainability of the scrubber.

16. The CO2 scrubber of claim 13, wherein a grain stabilizer in the porous monolithic is composed of at least one of ePTFE polymer, LPA polymer, PBSA polymer, alginate, and cellulose.

17. The CO2 scrubber of claim 13, wherein a grain stabilizer in the porous monolithic is composed of at least one biodegradable polymer such as PHA and PLA.
